(19) United States
(12) Patent Application Publication
(10) Pub. No.: US 2008/0009453 A1
(43) Pub. Date: Jan. 10, 2008

(54) Pharmceutical Compositions Containing Polydatin or Its Salts and Their Application

(75) Inventors: Jinhua Zhao, Nanshan (CN); Hui Kang, Nanshan (CN); Kesen Zhao, Nanshan (CN); Xuliang Huang, Nanshan (CN); Guiling Zhao, Nanshan (CN); Jing Li, Nanshan (CN); Hanlin Feng, Nanshan (CN); Guanghui Yao, Nanshan (CN); Qian Yao, Nanshan (CN); Shengzhao Lin, Nanshan (CN); Qi Zhou, Nanshan (CN)

Correspondence Address: RAYMOND Y. CHAN, 108 N. YNEZ AVE., SUITE 128, MONTEREY PARK, CA, 91754, US

(73) Assignee: Shenzhen Neptunus Pharmaceutical Co., Ltd., Nanshan (CN)

(21) Appl. No.: 10/492,405

(22) PCT Filed: Aug. 4, 2003
(86) PCT No.: PCT/CN03/00621

§ 371 Date: Apr. 4, 2005

(30) Foreign Application Priority Data
Oct. 8, 2002 (CN) 02134928.2

(51) Int. Cl. A61K 31/7034; A61P 7/00; A61P 9/00

(52) U.S. Cl. 514/35

(57) ABSTRACT

The invention discloses pharmaceutical compositions containing polydatin or its pharmaceutically acceptable salts with the effect of improving microcirculation, and their application in preparing drugs which can improve microcirculation. The compositions are an non-gastrointestinal injection preparation, the preparation can be an aqueous solution which have pH 7.0-10.0 comprising 5-50% propandiol. The dosage forms of the compositions may be lyophilizing powder injection, oral form, suppository of procto-administration and other forms of administrations comprising unguent, ointment, paste film coating and the like. The compositions may be used to treat and/or prevent diseases related to microcirculation blockage. The unit form may contain 1-1000 mg polydatin or its derivatives. The invention solves the problem of using polydatin to improve microcirculation. The composition of the invention can be used in treating shock, cardiac and cerebrovascular diseases, diseases of sense organs, diabetes complication, vaculitis related to blockage of blood vessel, hemorrhoid related to circulation obstruction, skin injury and burn.

### BACKGROUND OF THE PRESENT INVENTION

1. Field of Invention

The present invention relates to a pharmaceutical composition containing polydatin or its salts and/or acceptable vehicle for improving microcirculation, and further relates to the preparation and making of the pharmaceutical composition of the composition of the present invention and the function of the treatment or prevention of related diseases of occlusion of microcirculation through the application of the composition of the present invention. The composition of the present invention is capable of treating or preventing the obstructive microcirculation related diseases such as shock, myocardial ischemia, brain hypoxia, diabetes complication, glaucoma, obstructive microcirculation induced impaired hearing, hemorrhoid, thighbone ischemia, obstructive vasculitis, cirrhosis, chronic ulcer, and skin burn.

2. Description of Related Arts

Physiology researches show that the physiological function of the microcirculation mainly includes transporting oxygen, nutrients, physiological regulating active substances to cells, and removing metabolic wastes so as to maintain the normal activities of life. Many kinds of diseases are related to or arisen from the abnormal microcirculation, such as acute inflammatory diseases, traumatism, shock, chronic ulcer, cirrhosis, cardiovascular diseases, and diabetes mellitus. Therefore, improving the microcirculation is a very important and meaningful subject in the treatment and prevention of many diseases, and it is a highly recognizable subject in the medical and pharmaceutical industry for finding an effective medication for improving microcirculation.

Anisodamine hydrobromide, Atropine and Anisodine are the exemplary conventional medications for improving the microcirculation system, which share the common characteristics of having the effect of dilation of capillary, increasing the blood flow of tissues and organs. They are widely used in the treatment of different diseases.

Polydatin was first found in the plant Polygonum cuspidatum Sieb. Et Zucc. in the 60's in the 20 century (Nomura, 1963), and was then found in other plants such as peicin, grape and peanut. The chemical formula of polydatin is 3,4′,5-trihydroxy-trans-stilbene-3-b-d-glucose with the 3,4′,5-trihydroxy, also called resveratrol, as glucoside, is a stilbene compound. The chemical structure of polydatin is shown as follows:

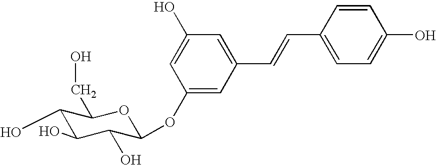

Since the 70's of the 20 century, a variety of researches of the pharmaceutical effect and activities of polydatin have been conducted in China. It is found that polydatin has the effect of lowering the cholesterol level of blood, that the use of 2.2 mg/kg/d polydatin is capable of lowering the total cholesterol, LDL/HDL cholesterol ratio, and blood platelet aggregation rate of a hyperlipoidemia patient (ZHANG, Pei Wen, 1995), the effect of inhibiting the aggregation of blood platelet and the formation of thrombus, that 6˜100 μmol/L polydatin is capable of inhibiting the production of the platelet accumulation and B₂ thrombus which are induced by AA and ADP (SHAN, Chun Wen, 1993), the effect of inhibiting blood platelet aggregation induced by adrenaline (YANG, Su Qin, 1992), the effect of inhibiting the blood platelet aggregation and the formation of thrombus caused by damage in common carotid artery in rabbit (by WANG, Yue Zhong, 1995), the effect of inhibiting the deformation and release reaction of blood platelet (by LIU, Lian Pu, 1998), the effect of inhibiting adhesive nature of white blood cells on inner wall of cell (by JIN, Chun Hua, 1998), the effect of strengthen the muscular strength (JIN, Xing Zhong, LUO, Su Fang, 1992), the effect of strengthen myocardial contraction and frequency (JIN, Chun Hua, 2000), the effect of vascular dilation such as the effect of promoting separated pulmonary artery dilation (LUO, Su Fang, 1992), the effect of contraction of main artery induced by non-competitive restrained platelet (WANG, Yue Zhong, 1994), the effect of anti-oxidation such as the inhibiting or removing function of polydatin on free radicals produced by PMNs of blood of human, Xanthin-Xanthin system, and VitC-Cu²⁺ system (JIN, Wei Jun, 1993), the effect of reducing damages to intestine and organs of rabbit (XIE, Zhong Lin, 1997), the effect of improving microcirculation and treatment of shock such as having treatment effect to shock caused by bleeding in rats which is better than that of using an equal dose of Dopamine and Anisodamine hydrobromide (HUANG, Qiao Bing, 1994), the effect of preventing and treating microcirculation of rats caused by burning (by WU, Kun Ying, 1992), the effect of reducing blood pressure difference of capillaries caused by bleeding and restoring the flowing of blood in capillaries (ZHU, Zuo Jiang, 1989), the effect of protecting damages to lungs caused by internal intoxication in rats (MO, Guo Yu, 1993), the effect of inhibiting increase in endothelin concentration of external blood flow induced by damages and decreasing the level of carbon monoxide (ZHAO, Wu Min, 1998), the effect of restoring and regulating body activities for burnt (XU, Chuan Lin, 1994). Furthermore, polydatin has liver protective function (HUANG, Zhao Sheng, 1998), and regulatory function in immune system (LUO, Rong Jiang, 1992).

Recently, many researches on the glucoside of polydatin, that is resveratrol, have been carried out and attracted international attention. ‘France paradox’ has been analyzed in 1992 and the cardiovascular protective function of resvertrol has been reported. Then, more and more documentations reported the pharmaceutical activity of resvertrol, such as, report of the anti-oxidative function and inhibiting effect on LDL peroxidation for preventing arteoclerosis (Pace-Asciak, 1996), and report on inhibiting the formation and developing of tumor (Jang M, Udeani GO et, Science 1997; 275: 218).

Recently, patent related documentations can also be found. For example, the PCT application WO 01/30336 A2 has disclosed the use of resvertrol for treating epidermal diseases, the PCT application WO 01/91714 A1 has disclosed the use of resvertrol in cosmetic products for treating dandruff or scurf, the PCT application WO 00/38620 has disclosed the use of resvertrol on treating periodontal diseases, the PCT application WO 00/12534 has disclosed the use of stilbene composition in treating hepatitis, the European patent EP 1138323 A2 has disclosed the use of resvertrol on treating eczema and psoriasis, the U.S. Pat. No. 6,008,260 A has disclosed the use of resvertrol on preventing tumor, the PCT application WO 00/44370 has disclosed the use of resvertrol derivative on treating osteoporosis and high blood pressure, the China patent 00121100.5 has disclosed the extraction of resvertrol and polydatin, the China patent 99115156.9 has disclosed the extraction of resvertrol from polydatin, the China patent 00113914.2 has disclosed the production method of cell having the composition resvertrol.

However, reports of composition of polydatin or its derivatives for improving microcirculation and treatment or preventing shock symptoms is not found. Moreover, the water solubility of polydatin is relatively poor which poses difficulties in the technical applications of polydatin in making composition for venous injection. Therefore, the polydatin used for venous injection is not found in different documentation in patent or patent applications.

### SUMMARY OF THE PRESENT INVENTION

A main object of the present invention is to provide a pharmaceutical composition having a quantity of polydatin or a quantity of salt of polydatin for improving the microcirculation. The pharmaceutical composition is capable of treating or preventing related obstructive diseases of microcirculation, wherein the composition comprising a quantity of polydatin or salt of polydatin is administered into a user.

Another object of the present invention is to provide a method of making a composition of polydatin or its salt for application on making a composition for improving microcirculation.

Accordingly, in order to accomplish the above objects, the present invention comprises a pharmaceutical composition capable of treating or preventing obstructive microcirculation related diseases, wherein the composition comprises an active composition of polydatin or a salt of polydatin, and one or more than one vehicle. The chemical formula of polydatin is 3,4′,5-trihydroxy-trans-stilbene-3-β-D-glucose.

The salt of polydatin is defined as a salt or an ester formed by polydatin and a predetermined acid including an organic acid or an inorganic acid, or and a predetermined alkaline including an organic or an inorganic alkaline.

One of the embodiments of the present invention is to provide a composition comprising polydatin or its salts which is applied externally, wherein the composition which is applied externally may be in the forms of solution, or a predetermined carrier such as injection solution, injection powder adapted for making an aqueous solution, or a frozen dry injection powder.

A preferred solvent of the aqueous solution of the present invention is physiological buffer or physiological saline which is applicable to a body. When making the aqueous solution for injection, the pH of the aqueous solution may be suitably adjusted such that the aqueous solution is neutral or alkaline under acceptable pharmaceutical conditions. For example, a range between pH 7.0 and pH 10.0 is capable of increasing the solubility of the active composition. The pH is preferably adjusted by using sodium carbonate (Na₂CO₃), sodium bicarbonate (NaHCO₃), or sodium hydroxide (NaOH) to adjust to pH 8.0 to 9.5, or by using a PBS buffer having a pH range between 8.0 and 9.5. In one of the embodiment of the present invention, the aqueous solution comprises 10 to 30% propandiol and 30 to 50% ethanol, and that the aqueous solution is diluted 25 to 50 times which is applicable to venous, muscular, or intravenous injection.

The aqueous solution of the present invention comprises a predetermined solubilizing agent, a lacquer solvent or a cosolvent for increasing the stability and solubility of the active composition of the present invention. For example, the aqueous solution comprises a predetermined quantity of low alcohol such as 5 to 20% 1,2-propandiol which is capable of increasing the solubility of the active composition of the present invention, and a predetermined quantity of superficial active agent such as 0.1 to 1% Tween 80 which is capable of increasing the stability of the aqueous solution. The solvent may further also be a cosolvent system comprising propandiol, manitol, non-polar superficial active agent, hydrated organic polymer, and cosolvent system in water phase.

A process of making the cool dry injection powder of the present invention comprises the step of providing an aqueous solution of the active composition of the present invention and cooling and drying under a suitable cool dry condition.

A composition of the present invention is capable of making a suspension solution by mixing lipophilic solvent or carrier and the active composition of the present invention. Examples of the lipophilic solvent or carrier include fatty oil or fatty acid ester such as triacylglycerol or liposome. The suspended solution may further comprise composition which increases the adhesion level such as sodium carboxyl methyl cellulose, sorbitol, or glucosans. A predetermined stabilizing agent or composition increasing solubility may be added for making concentrated aqueous solution.

The composition for external application preferably comprises the active composition having unit dose wherein the unit dose may comprise polydatin or its salt between the range of 1.0-1000 mg and the preferred unit dose is between 50 mg and 250 mg.

A second embodiment of the present invention is providing an oral application of the polydatin or its salts which is pharmaceutical acceptable.

The composition of the present invention for oral application may be in the form of tablet, pill, granule, capsule, solution, gel, syrup, and suspended solution.

A method of making the composition of the present invention for oral application may comprise a step of adding a predetermined quantity of vehicle such as microcrystallized cellulose, sucrose, lactose, glucose, starch, and manitol, binding agent such as syrup, Arabic gum, silica gel, sorbitol, tragacanth gum, methyl cellulose and polyethylene pyrrolidone; adding a predetermined quantity of disintegrating agent such as starch, carboxyl methyl cellulose and its calcium salt, microcrystalline cellulose, polyethylene glycol, cross-link polyethylene pyrrolidone, bubbler, alginic acid or its salt such as sodium alginate; adding a predetermined quantity of glidant such as talc powder, magnesium stearate, calcium stearate, and silica, lubricating agent such as magnesium stearate, sodium luarate, and glycerol, or the like. The method may also comprise the steps of mixing the active composition and a solid vehicle and grounding the mixed composition and vehicle to form a mixture, adding a predetermined auxiliary substance if necessary, and making the mixture into the form of a granule. The method may further comprise the steps of transforming the mixture in the form of a granule into the form of tablet or substance for coating with sugar, and the substance for coating with sugar is then properly coated by a coating materials such as concentrated sugar solution selected from the group comprising Arabia gum, talc powder, polyethylene pyrrolidone, resin of carboxyl polymer, polyethylene glycol, and/or titanium dioxide, lacquer solution, and a suitable organic solvent or solvent mixture. The mixture in the forms of tablet or pill may combine with a dye or color substance for distinguishing or representing a different dosage group.

The forms of oral administering composition further include capsule form made with gelatin, and soft capsule made from gelatin and plasticizing agent. The capsule form may comprise active composition and supplementary agent such as lactose, binding agent such as starch and/or lubricating agent such as talc or magnesium stearate, and stabilizing agent. The soft capsule form may comprise soluble active composition or suspension in suitable solution such as fatty oil or aqueous polyethylene glycol. Furthermore, stabilizing agent or lacquer solvent.

The oral administrative composition of the present invention is preferred to have a predetermined active composition in the quantity of unit dose, the unit dose can be polydatin or its salt in the range of 1.0 to 1000 mg, and preferably of 50 to 250 mg.

The composition of the present may also be in the form a rectal suppository comprising polydatin or its salt in the range of 1.0 to 1000 mg and a suppository agent such as coconut oil or other glyceride.

The composition of the present invention may also be localized administrative agent such as paste, cream, sticker, coating agent form comprising polydatin or its salt in the range of 1.0 to 1000 mg, and common supplementary substances such as Vaseline, glycerol, sodium lauryl sulfate, mono stearo glyceride, acetyl cellulose, bubblering oil, paraffin oil, and lanonine.

The method of administration of the composition of the present invention includes oral administration, eye drops administration, rectal suppository, membrane permeable administration, localized administration, or intestinal administration; external administration such as muscular, intravenous, marrow injection, and intrathecal or direct internal injection, venous abdominal or intraocular injection.

The composition of the present invention has significant dilation effect on capillaries, lowering the blood viscosity level of capillaries, inhibiting the blood cell aggregation, increasing the microcapillaries dilation rate, and hence have significant effect on improving the microcirculation and is applicable in treatment or preventive measure of microcirculation obstructive disease.

The composition of the present invention can also be used for preventing or treating shock.

One of the symptoms of shock is microcirculation obstruction which induces insufficient irrigation or circulation of blood to important organs and may result in deterioration of function of organs. The active composition of the composition of the present invention is capable of exerting dilating effect to the microvascular vessels of animals, increasing the opening rate of capillaries of organs, increasing difference in blood pressure, thus is capable of effectively improving the blood circulation for different organs under shocking conditions and increasing the animal survival number in the shock model experiment.

In the treatment for shock, venous administration is preferred such as venous injection or instillation. The dosage of the composition depends on the body weight, age, body conditions and is decided by a practitioner. For an adult, a preferred suitable dose is about 20 to 600 mg per dose, preferably 80 to 300 mg per dose.

The present invention is capable of using for treating or preventing cardiovascular and cerebral vascular diseases.

Cardiovascular and cerebral vascular diseases are usually related to microcirculation obstruction. For example, cerebral hypoxia, insufficient blood flow resulted from insufficient supply of blood. The active composition of the composition of the present invention can improve the microcirculation and thus improve the blood flow to heart and brain such that the aim for treating or preventing cardiovascular and cerebral vascular diseases is achieved.

When the composition of the present invention is used for treating or preventing vascular diseases, the composition can be in the form of oral administrable medication or medication for external use. The appropriate dosage is decided by practitioner according to the weight, age, body conditions of the patient. The appropriate dosage for an adult is 20-600 mg per day which can be applied in a single dose or divided into several doses. The preferable dose is 20-80 mg per application, at a frequency of 2-3 doses for day.

The present invention is capable of using as a medication for treatment and preventive measure for some of the obstructive diseases of sense organs.

Some of the obstructive diseases of sense organs such as visual acuity decreases from microcirculation obstruction, or deaf caused by microcirculation obstruction. The active composition of the pharmaceutical composition of the present invention can improve the circulation of these sense organs and thus treating and preventing the visual and hearing obstructive diseases.

When the composition is used for treating or preventing visual and hearing diseases in eye and ear, the applications of the composition of the present invention can be applied as system administration such as oral administration or venous administration, and as localized administration such as eye dropping and intraocular injection. The appropriate dosage is decided by practitioner according to the weight, age, body condition of the patient. In the application as system administration, the appropriate daily dosage is between 20 and 600 mg which can be applied as a single dose or divided into separated dose.

The composition of the present invention can also be used for treating or preventing other related diseases of microcirculation obstruction. For example, the composition of the present invention can be used for venous thrombosis, external hemorrhoid caused by blood clot, skin damage or burn. The application of treating and preventing these diseases by the composition of the present invention can be system administration or localized administration.

The present invention is first using polydatin composition for improving the microcirculation in the pharmaceutical mediation, such that can be used for treating and preventing a variety of microcirculation obstructive diseases. In those intravenous medication, since the solubility of polydatin is relatively poor, under normal solubility condition and medication volume, it is difficult to provide an effective quantity of active composition in its aqueous solution. The present invention comprises alkaline solvent and solubilizing agent for effectively increasing the solubility of polydatin.

These and other objectives, features, and advantages of the present invention will become apparent from the following detailed description, the accompanying drawings, and the appended claims.

### DETAILED DESCRIPTION OF THE PREFERRED EMBODIMENT

According to a first embodiment of the pharmaceutical composition of the present invention, a process of making polydatin aqueous solution for injection is provided.

According to a first exemplary illustration of the first embodiment, a process of making an aqueous solution for injection is described, wherein the aqueous solution has a composition comprising a quantity of polydatin, a quantity of NaOH aqueous solution at pH 8.5 and a quantity of 3.6% NaCl aqueous solution. According to the composition of the first exemplary illustration, the quantity of polydatin is 40 g, the quantity of NaOH solution is 8 L, and the quantity of 3.6% NaCl is used for adjusting the total volume of the composition into a 10 L solution, wherein the composition of the first exemplary illustration is divided into 1000 unit.

The process of making an aqueous solution for injection comprises the steps of dissolving 40 g polydatin in 8 L NaOH aqueous solution at pH 8.5; adding a quantity of NaCl until the total volume is 9.8 L; adjusting the pH to 8.5 and adding NaCl aqueous solution until the total volume is 10 L; filtering using 0.22 μm microfilter membrane; and dividing the solution into 1000 units and keeping in ampoules or bottles.

### EMBODIMENT 1-2

According to a second exemplary illustration of the first embodiment, a process of making an aqueous solution for injection is described, wherein the aqueous solution has a composition comprising a quantity of polydatin, a quantity of NaOH aqueous solution at pH 8.5, a quantity of propandiol, a quantity of Tween 80, and a quantity of 3.6% NaCl aqueous solution. According to the composition of the first exemplary illustration, the quantity of polydatin is 80 g, the quantity of NaOH solution is 6 L, the quantity of propandiol is 2 L, the quantity of Tween 80 is 50 ml, and the quantity of 3.6% NaCl is used for adjusting the total volume of the composition into a 10 L solution, wherein the composition of the first exemplary illustration is divided into 1000 unit.

The process of making an aqueous solution for injection in the embodiment 1-2 comprises the steps of dissolving 40 g polydatin in 2 L propandiol solution; adding 50 ml Tween 80 and NaOH aqueous solution respectively and mixing; adding a quantity of 3.6% NaCl until the total volume is 9.8 L; adjusting the pH to 8.5 and adding NaCl aqueous solution until the total volume is 10 L; filtering using 0.22 μm microfilter membrane; and dividing the solution into 1000 units and keeping in ampoules or bottles.

### EMBODIMENT 1-3

According to a third exemplary illustration of the first embodiment, a process of making an aqueous solution for injection is described, wherein the aqueous solution has a composition comprising a quantity of polydatin, a quantity of anhydrous alcohol, propandiol, and a quantity of sodium carbon. According to the composition of the third exemplary illustration, the quantity of polydatin is 100 g, the quantity of anhydrous alcohol is 2250 ml, the quantity of propandiol is 1000 ml, and the quantity of sodium carbonate is used for adjusting the total volume of the aqueous solution into 5000 ml. The aqueous solution in the embodiment 1-3 is diluted 25 to 50 times by 0.9% sodium fluoride or 5% glucose injection solution for administration.

The process of making an aqueous solution for injection in the embodiment 1-3 comprises the steps of dissolving 20 g polydatin in 450 ml anhydrous alcohol; adding 100 ml buffer solution and mixing by ultrasound or stirring for 2 to 5 minutes such that the polydatin is dissolved; adding 200 ml propandiol and the remaining buffer solution until the total volume is 1 L; filtering using 0.22 μm microfilter membrane; and refilling nitrogen and dividing the solution into 1000 units which are kept in brown ampoules or bottles. The buffer solution is prepared by mixing one unit of 0.1 mol/L sodium carbonate and nine units of 0.1 mol/L sodium hydrocarbonate.

### EMBODIMENT 2

A process of making a cool dry powder of polydatin for injection.

### EMBODIMENT 2-1

A process of making a polydatin solution is illustrated in embodiment 2-1. The process of making a polydatin solution in the embodiment 2-1 comprises the steps of dissolving 40 g polydatin in 8 L pH 8.5 NaOH aqueous solution; adding a quantity of 5% aqueous manitol solution until the total volume is 9.8 L; adjusting the pH to 8.5 and adding 3.6% NaCl aqueous solution until the total volume is 10 L; filtering using 0.22 um microfilter membrane; and dividing the solution into 1000 units and keeping in vial for dry powder.

A process of freeze dry is also illustrated in the embodiment 2-1, comprising the step of obtaining the solution obtained from the process of making a polydatin solution, and placing the solution in a freeze dry condition with a temperature at −35° C. such that the solution is kept in −30° C. for 3 hours; vacuum pumping at condensation temperature at −40° C. and increasing the temperature to 40° C. gradually such that the frozen solution is increased gradually; drying the frozen solution and that the freeze dry product is obtained. The product can then be packed and pressed with piston, enclosed and labeled as a polydatin injection powder. The powder is adapted for dissolving in physiological saline for injection use.

### EMBODIMENT 2-2

A process of making a polydatin solution is illustrated in embodiment 2-2. The process of making a polydatin solution in the embodiment 2-2 comprises the steps of dissolving 80 g polydatin in 8 L pH 9.5 NaOH aqueous solution; adding a quantity of 5% aqueous manitol solution until the total volume is 9.8 L; adjusting the pH to 8.5 and adding 3.6% NaCl aqueous solution until the total volume is 10 L; filtering using 0.22 μm microfilter membrane; and dividing the solution into 1000 units and keeping in vial for freeze dry powder.

A process of freeze dry is also illustrated in the embodiment 2-2, comprising the step of obtaining the solution obtained from the process of making a polydatin solution, and placing the solution in a freeze dry condition with a temperature at −30° C. such that the solution is kept in −30° C. for 3 hours; vacuum pumping at condensation temperature at −40° C. and increasing the temperature to 40° C. gradually such that the frozen solution is increased gradually; drying the frozen solution and that the freeze dry product is obtained. The product can then be packed and pressed with piston, enclosed and labeled as a polydatin injection powder. The powder is adapted for dissolving in physiological saline for injection use.

A process of making an injection solution of the present invention is illustrated in the embodiment 2-2, comprising the steps of mixing 2 L propandiol and 500 ml 0.5% hydrochloric acid; adding a predetermined quantity of physiology saline such that the total volume is 10 L, filtering using 0.22 μm microfilter membrane; and dividing the solution into 1000 units and keeping in vial. Each of the vials comprises 10 ml of injection solution.

A package comprising a 10 ml injection solution and a unit of freeze dry powder is preferred such that the freeze dry powder is capable of dissolving in the injection solution for injection.

### EMBODIMENT 3

A process of making a composition of polydatin in tablet form. 1000 tablet each having a composition of 50 mg polydatin are prepared according to the routine making process, and that the composition of polydatin comprises a quantity of polydatin, a quantity of lactose, a quantity of starch, a quantity of polyethylene pyrrolidone K30, and a quantity of magnesium stearate. In embodiment 3, 50 g of polydatin, 107 g of lactose, 25 g of starch, 16 g of polyethylene pyrrolidone K30, and 2 g of magnesium stearate are used and that the total weight is 200 g.

The polydatin is mixed with lactose and starch to form a mixture. The mixture is transformed into a granule form by using polyethylene pyrrolidone K30 and passing through a No. 16 model unit. After drying, the granule is mixed with magnesium stearate and pressed into a predetermined shape. Other polydatin in tablet form having different active composition of polydatin can be made by varying the ratio of the quantity of polydatin and vehicle, or the force of pressing device.

### EMBODIMENT 4

A process of making a composition of polydatin in capsule form, comprising the steps of: mixing a quantity of polydatin, a quantity of lactose, a quantity of microcrystalized cellulose, and a quantity of magnesium stearate. In the embodiment 4, 200 capsules having 50 mg active composition of polydatin are prepared from 10 g polydatin, 19.5 g lactose, 10 g microcrystalized cellulose, and 0.5 g magnesium stearate, wherein a total weight is 40 g.

### EMBODIMENT 5

A process of making a composition of polydatin in ointment form, comprising the steps of: mixing and stirring a quantity of polydatin and a quantity of olive oil; and adding a quantity of dissolved white Vaseline while stirring; and mixing thoroughly. The ointment is then canned. In embodiment 5, a 1% polydatin ointment is prepared from 1 g polydatin, 10 g olive oil, and 89 g white Vaseline wherein the total weight is 100 g.

### EMBODIMENT 6

A process of making a composition of polydatin in suppository form, comprising the steps of: mixing and stirring a quantity of polydatin and a quantity of glycerol; and adding a quantity of dissolved glycerol gelatin while stirring; and mixing thoroughly. The suppository is then canned. In embodiment 5, a 1% polydatin suppository is prepared from 1 g polydatin, 5 g glycerol, and 84 g glycerol gelatin wherein the total weight is 100 g.

### EMBODIMENT 7

A treatment action for shock related to blood loss of a composition of polydatin.

The observation is based on the experiment carried out comprising the steps of

(1) anaesthetizing two groups, namely a treatment group and a control group, of SD rats with Urethane Ketamine, inducing bleeding by intubulation at femoral artery and recording blood pressure; wherein femoral vein is used for medication and blood transfusion and cremasteric specimen is prepared by Baez method;

(2) inducing bleeding from femoral artery such that an average arterial blood pressure is maintained between 5.1 to 5.6 kPa;

(3) administering a composition of polydatin injection through femoral vein after one hour wherein the dosage of the composition is 0.6 ml/kg, that the concentration of polydatin is 4 mg/ml and the dosage is 2.4 mg/kg in the treatment group and the composition of polydatin is replaced by physiological saline in the control group;

(4) conducting blood transfusion after 20 minutes such that the volume of blood loss due to bleeding is equal to the volume of blood loss due to blood transfusion; and

(5) observing for two hours and detaching the intubulation.

The records are made by using Olympus microscope, Hitachi monitoring system and physiological recording meter to observe and record microcirculation and blood dynamics. The survival period of each subject of the two groups are recorded.

Result:

(1) Survival period: a ratio of the survival time of the treatment group and the control group is larger than 5.5, wherein 9/10 of the treatment group has a survival period of 24 hours, 8/10 of the treatment group has a survival period of 48 hours, and 6/10 of the treatment group has a survival period of 72 hours. All the subjects in the control group die within 24 hours.

(2) Blood pressure: After experienced shock for 1 hour, the average arterial blood pressure is 5.3+0.2 kPa. After 30 minutes of medication of polydatin, the arterial pressure is increased to 8.6+0.8 kPa in the treatment group, while there is no significant difference in blood pressure in the control group receiving physiological saline.

(3) Diameter of micro blood vessel: After experienced shock for 1 hour, the diameter of micro-aneurysm is reduced to 63.3% of the diameter in average before shock. The diameter is then increased by 20.3% after medication of polydatin and there is no significant difference of diameter in the control group.

(4) Number of opening capillaries: Before shock, the number of open capillary is 5.1±0.4 per μm². After induced bleeding, the number of open capillary is decreased to 2.2±0.6 per μm². The number increased to 4.2±0.5 per μm² in 10 minutes after polydatin medication. There is no significant difference of number of opening capillaries in the control group before and after the administration of physiological saline.

### EMBODIMENT 8

A treatment action for shock related to skin burn of a composition of polydatin.

The observation is based on the experiment carried out comprising the steps of: anaesthetizing two groups, namely a treatment group and a control group, of SD rats with Urethane Ketamine, burning about 35% of body surface below waist for 30 seconds with 80° C. water; administering 0.6 ml/kg polydatin injection (comprising 4 mg/ml polydatin and the dosage is 2.4 mg/kg) by venous injection after half hour of burning in the treatment group and replacing the polydatin injection by physiological saline in control group.

Result: The average survival period of the treatment group is longer than 13.3 hours while the average survival period of the control group is 7.0±2.6 hours.

One skilled in the art will understand that the embodiment of the present invention described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims.

1-16. (canceled)

17. A composition having the effect of improving the microcirculation, comprising:
an effective amount of active ingredient consisting of polydatin or its pharmacologically acceptable salt; and
pharmaceutically acceptable carrier and vehicle such that said carrier carries said active ingredient and said vehicle provides a medium for said active ingredient;
wherein said polydatin has a chemical formula of 3,4′,5-trihydroxy-trans-stilbene-3-β-mono-D-glucoside and a chemical structure:

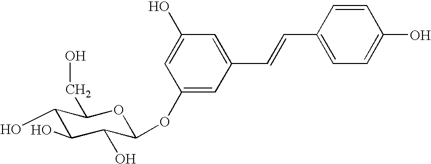

18. The composition, as recited in claim 17, having an injection form applicable for administration by injection.

19. The composition, as recited in claim 18, wherein said injection form is an aqueous solution.

20. The composition, as recited in claim 19, wherein said aqueous solution has a pH in the range between 7.0 and 10.0.

21. The composition, as recited in claim 19, wherein said aqueous solution has a pH in the range between 8.0 and 9.5.

22. The composition, as in claim 19, 20 or 21, further comprising a quantity of propandiol having a concentration in the range between 5% and 50%.

23. The composition, as in claim 19, 20 or 21, further comprising a quantity of propandiol having a concentration in the range between 10% and 30% and a quantity of ethanol having a concentration in the range between 30% and 50%, wherein said injection is adapted to be diluted 25 to 50 times for injection selected from the group of administration consisting of intravenous injection, intramuscular injection, and hypodermis injection.

24. The composition, as recited in claim 18, wherein said composition has a lyophilized powder form.

25. The composition, as recited in claim 17, wherein said composition has an administration form selected from the group consisting of tablet, capsule, suppository, ointment, cream, sticker, and paste.

26. The composition, as in claim 18, wherein said composition is prepared by a manufacturing process comprising the steps of:
(a) dissolving a 40 g polydatin in a 8 liter sodium hydroxide. aqueous solution to form a starting solution having a pH 8.5;
(b) adjusting a volume of said starting solution to 9.8 liter by adding 3.6% sodium chloride;
(c) adjusting a pH of said starting solution to 8.5 and adding 3.6% sodium chloride for adjusting said volume of the starting solution to 10 liter; and
(d) filtering said starting solution using a 0.22 um microfilter membrane to obtain a final solution having 1000 units,
wherein said composition is adapted for treating and preventing microcirculation obstruction related diseases of shock, cardiac and cerebral vascular disease, sensory organ related disease, diabetes complications, vasculitis related to blockage of blood vessel, hemorrhoid related to microcirculation obstruction, skin injury and burn.

27. The composition, as in claim 21, wherein said composition is prepared by a manufacturing process comprising the steps of:
(a) dissolving a 40 g polydatin in a 2 liter propandiol aqueous solution and mixing with a 50 ml Polyoxyethylene sorbitan monooleat and a 50 ml 3.6% sodium hydroxide solution to form a starting solution;
(b) adjusting a volume of said starting solution to 9.8 liter by adding 3.6% sodium chloride;
(c) adjusting a pH of said starting solution to 8.5 and adding sodium chloride for adjusting said volume of the starting solution to 10 liter; and
(d) filtering said starting solution using a 0.22 μm microfilter membrane to obtain a final solution having 1000 units,
wherein said composition is adapted for treating and preventing microcirculation obstruction related diseases of shock, cardiac and cerebral vascular disease, sensory organ related disease, diabetes complications, vasculitis related to blockage of blood vessel, hemorrhoid related to microcirculation obstruction, skin injury and burn.

28. The composition, as in claim 21, wherein said composition is prepared by a manufacturing process comprising the steps of:
(a) dissolving a 20 g polydatin in a 450 ml anhydrous alcohol and mixing with a 100 ml buffer solution until the polydatin is dissolved to form a starting solution;
(b) adding 200 ml propandiol before adjusting a volume of said starting solution to 1 liter by adding the buffer solution; and
(c) filtering said starting solution using a 0.22 μm microfilter membrane to obtain a final solution having 1000 units,
wherein the buffer solution has a ratio of 0.1 mol/L sodium carbonate to 0.1 mol/L sodium hydrocarbonate 1:1,
wherein said composition is adapted for treating and preventing microcirculation obstruction related diseases of shock, cardiac and cerebral vascular disease, sensory organ related disease, diabetes complications, vasculitis related to blockage of blood vessel, hemorrhoid related to microcirculation obstruction, skin injury and burn.

29. The composition, as in claim 24, wherein said composition is prepared by a manufacturing process comprising the steps of:
(a) dissolving a 80 g polydatin in a 8 liter sodium hydroxide aqueous solution to form a starting solution having a pH 9.5;
(b) adjusting a volume of said starting solution to 9.8 liter by adding 5% aqueous manitol solution;
(c) adjusting a pH of said starting solution to 8.5 and adding 3.6% sodium chloride for adjusting said volume of the starting solution to 10 liter; and
(d) filtering said starting solution using a 0.22 um microfilter membrane to obtain a final solution having 1000 units, (e) placing the final solution at −30° C. for three hours, vacuum pumping at −40° C. and gradually increasing the temperature to 40° C. to obtain a frozen solution,
(f) drying the frozen solution to form a powder form,
wherein said composition is adapted for treating and preventing microcirculation obstruction related diseases of shock, cardiac and cerebral vascular disease, sensory organ related disease, diabetes complications, vasculitis related to blockage of blood vessel, hemorrhoid related to microcirculation obstruction, skin injury and burn.

30. The composition, as in claim 18 or 25, further having a quantity of active composition selected from the group consisting of polydatin and salt of polydatin having the same activity as said polydatin in an unit dose having a quantity range between 1 mg and 1000 mg.

31. The composition, as in claim 18 or 25, having the effect of improving microcirculation, wherein said composition is an active substance capable of being used in a manufacturing process of pharmaceutical composition for improving microcirculation.
